(19) United States
(12) Patent Application Publication
(10) Pub. No.: US 2008/0319393 A1
(43) Pub. Date: Dec. 25, 2008

(54) Self-Powered Portable Syringe Pump

(75) Inventor: Kirk Elder, West Palm Beach, FL (US)

Correspondence Address: Margaret J. McLaren, P.A.;McLaren Legal Services, 6500 S.W. 133rd Drive, Miami, FL, 33156, US

(73) Assignee: ANTLAE LLC, Fort Lauderdale, FL (US)

(21) Appl. No.: 12/088,171

(22) PCT Filed: Jan. 6, 2006
(86) PCT No.: PCT/US06/00412

§ 371 Date: Jul. 14, 2008

Related U.S. Application Data
(60) Provisional application No. 60/720,759, filed on Sep. 27, 2005.

(51) Int. Cl. A61M 5/20

(52) U.S. Cl. 604/151

(57) ABSTRACT

A disposable self-powered portable syringe pump (100) has been developed for use with syringe-based systems for delivery of medicinal solutions and other fluids. The syringe pump is lightweight, inexpensive to manufacture, easily adjustable and compatible with a wide variety of syringes in use around the world.

### CROSS-REFERENCE TO RELATED APPLICATIONS

The present application claims priority to U.S. Provisional Patent Application Ser. No. 60/720,759 entitled “Portable Compact Syringe Pump,” filed Sep. 27, 2005, the disclosure of which is hereby incorporated by reference in its entirety.

### BACKGROUND OF THE INVENTION

The invention relates generally to the field of fluid infusion devices for medical applications. More specifically, it relates to self-powered pumping devices used for the delivery of a liquid medication from a syringe into an intravenous (IV) administration system.

Various devices allow for IV infusion of liquid medications into a patient at a controlled flow rate over an extended period of time. Gravity flow IV administration has been employed for many years, in which a container such as a bag of liquid medication is typically hung from a metal pole provided with casters for mobility. More recently, IV systems having pumps powered by electricity or other means have been developed.

Medical facilities of all kinds face continued pressure to contain costs, reduce personnel and shorten the duration of in-patient care, while continuing to provide state-of-the-art therapies for their patents. Many existing IV systems are bulky and costly to manufacture and were designed primarily for use in bedridden patients under supervision and control of medically-trained personnel and assistants, typically in a hospital setting.

Unfortunately, there are numerous applications both within modern medical facilities and in many other settings in which medical services are administered for which one or more features of known infusion devices render them unsuitable, impractical, or prohibitively expensive to use. For example, it requires two assistants to transport a bedridden patient attached to a typical gravity-fed IV system—one to move the bed and the other to guide the IV pole from one location to another. Additionally, if a patient is being transported within a medical setting, for example from a hospital room to a facility providing an imaging procedure such as MRI or CT scan, or a therapy such as hyperbaric oxygen treatment, a further complication is the need to avoid the presence of metallic parts, or the generation of electrical emissions within these imaging or treatment units. This can necessitate ceasing the IV administration during the imaging or treatment procedure to avoid interfering with the proper functioning of the equipment, or a time-consuming rearrangement of machine-incompatible parts of the infusion devices or ancillary equipment, such as IV poles, electric pumps, batteries and like.

Medical situations and conditions calling for infusion of medicaments also occur in a great variety of settings outside of traditional medical facilities, and further underscore the need for improvement in infusion devices, particularly with regard to portability and flexibility of use. For example, personnel such as paramedics working in mobile medical facilities, first responders to emergencies and disasters, and military personnel must contend with space and other constraints generally not encountered in permanent medical facilities. In such situations, doses of IV medications may be provided and most efficiently stored in prefilled syringes of particular sizes. The prefilled syringes are activated by pump devices that must be compatible with the particular syringe size in order to dispense the medication by continuous infusion. Lack of availability of a pump device of the correct size to fit the syringe can render the syringe useless, except for acute administration of the medication.

Furthermore, in many emergency situations such as on a battlefield or after a natural or manmade disaster, IV medications must be administered to victims in absence of access to power, remote from any medical facility and with a minimum of assistance from others. Such dire circumstances call for self-powered portable infusion pumps that are compatible with pre-filled syringes in a full range of sizes that are lightweight for portability, and easy to use under the most challenging of circumstances.

Therefore both within medical facilities and in the field there continues to be an unmet need for compact, self-powered syringe pump devices adaptable for use with an IV administration syringe, that are lightweight, free of metal parts, compatible with all sizes of IV syringes, economical to manufacture, and easy and convenient to use.

### BRIEF SUMMARY OF THE INVENTION

Broadly, the invention provides a novel portable self-powered pump device for delivering the contents of a fluid-filled syringe such as an intravenous (IV) administration syringe. The self-powered pump device incorporates many advantageous features over existing infusion devices. Power is provided to the pump by elastomeric members coupled between the body of the device and a cap that, when engaged with a syringe contained therein, exert force on the plunger of a fluid-filled syringe to dispense the contents uniformly and repeatedly over the entire stroke length of the plunger, for up to 1000 uses. Preferred embodiments are configured with particularly advantageous features such as an adjustable body suitable for accommodating the barrel of any size of syringe, a self-centering cap that is adapted to fit many sizes and shapes of syringe plungers. Constructed of lightweight, inexpensive materials, the device can be manufactured at sufficiently low cost to permit disposable use, but is robust enough to withstand sterilization and multiple use if desired.

Accordingly, and in one aspect, the invention provides a self-powered portable pump device for dispensing fluid content from a syringe, comprising: a cap adapted to receive a plunger of a syringe; a body adapted to hold the barrel of a syringe; and at least two elastomeric members for operatively coupling the cap to the body, to apply a force to the plunger of the syringe for dispensing fluid content from the syringe. Further provided are kits comprising syringe pumps of the invention and one or more syringes pre-filled with a liquid medication.

These and other objects, advantages, and features of the invention will become apparent to those persons skilled in the art upon reading the details of the invention as more fully described below.

### BRIEF DESCRIPTION OF THE DRAWINGS

FIG. 1 is a perspective drawing of a portable self-powered syringe pump, in accordance with the invention;

FIG. 2 is a perspective drawing of a portable self-powered syringe pump shown in an open position surrounding an intravenous administration syringe, being loaded therein in accordance with the invention;

FIG. 3 is a perspective drawing illustrating a step in the adjustment of a portable self-powered syringe pump in accordance with the invention; and

FIG. 4 is a perspective drawing showing the pump in use in accordance with the invention.

FIG. 5 is a schematic diagram of a cover for use with a syringe pump in accordance with the invention.

### DETAILED DESCRIPTION OF THE INVENTION

The invention generally provides a novel portable self-powered pump device for delivering the contents of a fluid-filled syringe, such as a standard IV administration syringe, for liquid medications to human or animal subjects.

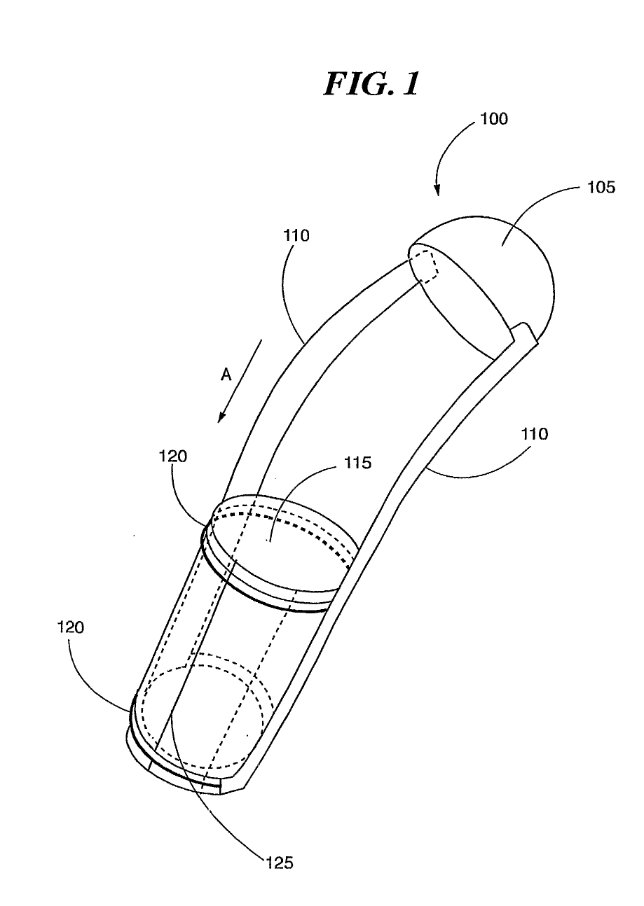

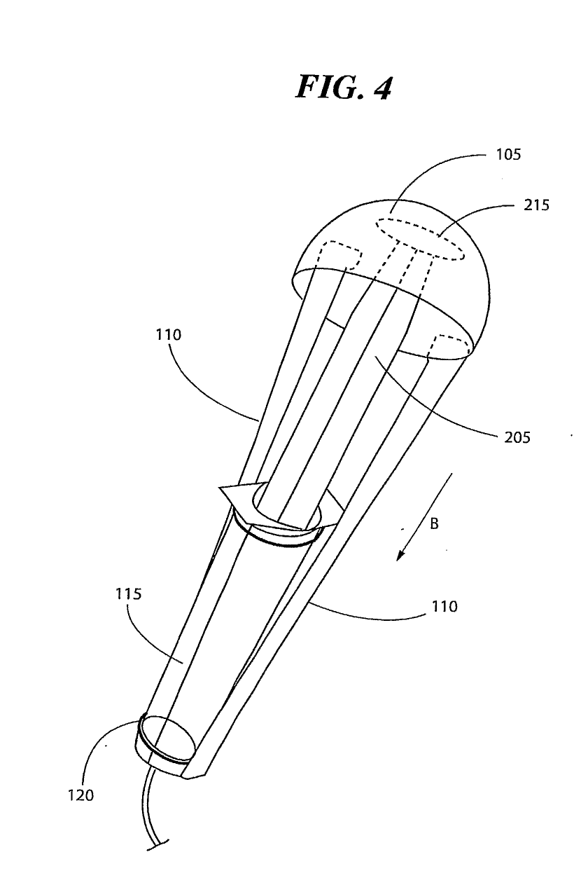

Referring now to the drawings, a portable self-powered syringe pump 100, in accordance with the present invention, is shown. Referring to FIG. 1, pump 100 includes a self-entering cap 105 adapted to receive a plunger of a syringe (as shown in FIG. 4), a body 115 configured in the form of an adjustable sleeve for holding the barrel of a syringe, and at least two elastomeric members 110 for operatively coupling the cap 105 to the sleeve 115 to apply an elastic or restorative force between cap 105 and sleeve 115. In other words; if deformed by being moved away from sleeve 115, elastic members 110 will apply a force in the direction of arrow A pulling cap 105 towards sleeve 115.

Powering the pump device 100 are elastomeric members 110. Elastomeric members 110, when deformed, provide power capable of delivering the contents of the syringe into the body of the subject by means of an IV infusion system (not shown) connected proximally to the outlet tip of an IV administration syringe and distally to the interior of a vein or artery or other body part of a subject according to standard medical procedures and systems in use for IV administration of fluids. Suitable IV infusion syringes and systems are well known in the art. Typically, the outlet tip of the IV syringe is attached to the infusion tubing by a Luer lock connection, to prevent accidental disconnection of the tubing during perfusion. The flow rate into the body structure (typically a blood vessel) of the human or animal subject is controlled by a regulator device connected to the tubing and positioned between the outlet tip of the syringe and the site of entry of the perfusion system into the subjects body.

Elastomeric members 110 are fixed across a gap, between opposed sides of cap 105 and opposed sides of sleeve 115. When elastomeric members 110 are deformed by separating cap 105 from sleeve 115, the elastic restorative properties cause it to provide a potential force in the direction of arrow A, when as discussed below, a syringe 205 is placed within pump 100, a force is applied to the syringe plunger in the direction of arrow A along a stroke length, corresponding to the length of deformation applied to elastomeric members 110. In a preferred but non-limiting embodiment, elastomeric members apply a constant force along the stroke length to ensure that the medicament within the syringe is applied consistently throughout its application. Furthermore, in a preferred non-limiting embodiment the elastomeric members 110 consistently return nearly 100 percent of the energy invested in them for over 1,000 cycles of deformation and return.

In a preferred, non-limiting example, elastomeric members 110 are fabricated from silicone-fortified polyurethane compounds which were tested and determined to apply a substantially constant force along the entire stroke length of the plunger of the syringe.

In one preferred embodiment, the compound used for the elastomeric members 110 comprises a mono polyurethane induction, such as mono n-butylamine. Suitable products are sold under the trade names E-Last™ and Poly-U-Last™ (Aged to Perfection, Fort Lauderdale, Fla.).

Properties of an exemplary sampling of a silicone-fortified polyurethane compound suitable for use in the invention are as follows:
1. Physical Properties:
   specific gravity; 1.13, % volatile <0.5% by weight
2. Viscosity:
   100000.0 centipoise
3. Reactivity Data—hazardous decomposition products:
   carbon monoxide, oxides of nitrogen, hydrogen cyanide
4. Stability:
   stable
5. Hazardous Polymerization:
   will not occur
6. Voc:
   0.01 lbs/gat calculated, voc; 2 gms./liter
7. Tensile Test
   A ⅛ inch (0.3175 cm) dumbbell specimen with a ⅛ inch (0.3175 cm) square cross section was tested at 2.0 inches/minute (5.08 cm/minute)

| | | Tensile Strength | |
|---|---|---|---|
| Relative Humidity | Temperature | psi (kg/cm┘) | Elongation % |
| 50% | 70° F. (21° C.) | 700 (49.21) | 1350 |

Self-centering cap 105 has a convex hemispherical shape adapted to receive the end of a syringe plunger therein. The convex hemispherical shape is symmetrical. Therefore, as elastomeric members 110 apply a force in the direction of Arrow A on cap 105, this will cause cap 105 to slide along the end of the syringe plunger until the forces are equalized at each side of cap 105 relative to the syringe plunger. In other words, where the syringe plunger contacts the cap substantially equidistant or symmetrical relative to a central point of the hemisphere. In other word, it is self-seeking to the point where cap 105 provides a balanced force on the syringe plunger.

The invention is not limited to a hemispherical shape. Other symmetrical or asymmetrical shapes can be used for the self-centering cap 105, provided that the cap is appropriately shaped such that a uniform pressure is transmitted to the syringe plunger by the elastomeric members 110 attached to the cap 105 and the cap will slide along the plunger until that point is found.

In a preferred embodiment, sleeve 115 forms an adjustable body suitable for holding syringe barrels of substantially any size or shape. In the particular embodiment shown in FIG. 1, the adjustable body is configured in the form of a sleeve 115 comprising a flexible sheet of non-metallic material such as polycarbonate, folded upon itself in a roll or tube shape with overlapping ends 125, as seen for example in FIG. 1. One or more elastic members 120 (two shown) provide support for the rolled shape of sleeve 115 and are used to adjust and maintain the diameter of sleeve 115 after insertion of a syringe into the device 100.

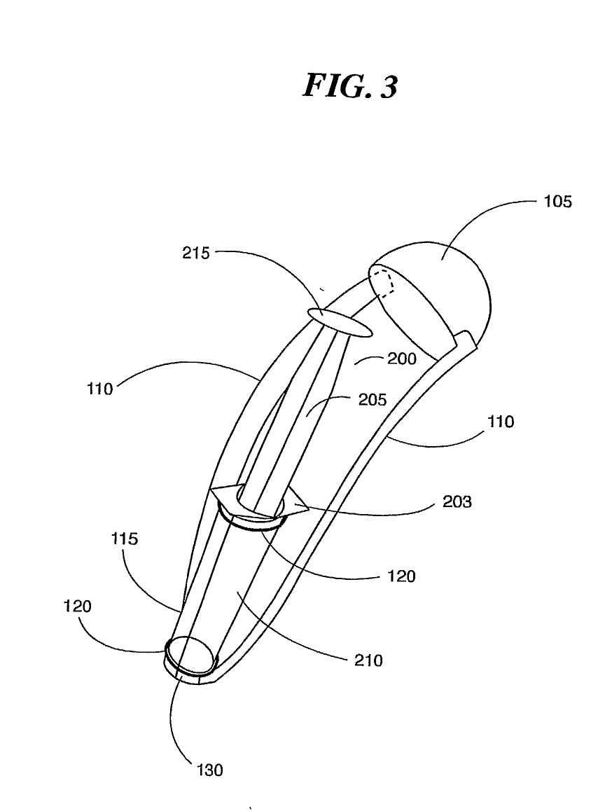

In a preferred, non-limiting example, elastic members 120 are formed as O-rings to facilitate use with the cylindrical shape of sleeve 115. Referring again to FIG. 1, self-centering cap 105 and adjustable sleeve 115 are fabricated from non-metallic material that is substantially transparent to light, electronic, and magnetic transmissions. The non-metallic material is a sturdy resilient material such as a flexible and waterproof material that can be formed into thin sheets and can withstand the temperature and humidity of an autoclave. In a preferred, non-limiting example, plastic materials are used and a particularly preferred plastic material is a polycarbonate. A translucent or transparent polycarbonate material is particularly useful for visualization of the plunger and barrel of a syringe positioned inside the pump, as shown in FIGS. 3 and 4.

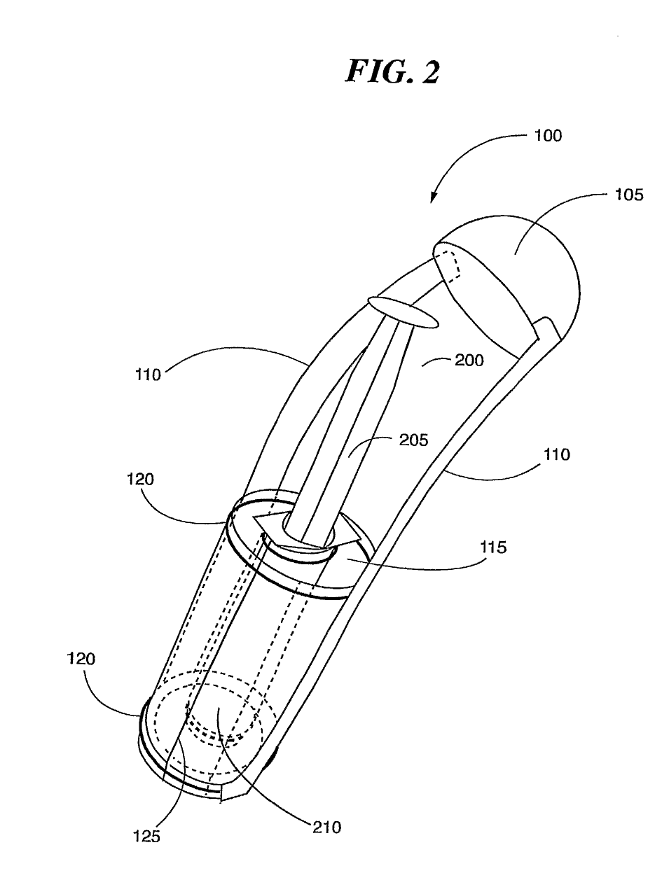

Reference is now made to FIGS. 2-4 in which operation of pump 100 is shown. FIG. 2 schematically illustrates a portable infusion pump device 100 of the invention with a typical infusion syringe 200 having a plunger 205 and barrel 210 loosely inserted inside the pump 100 (elastic members 120 are stretched in this view so that an inner diameter of sleeve 115 is enlarged to receive barrel 210). Adjustable sleeve 115 is tightened around the syringe barrel 210 as shown in FIG. 3. By comparing FIGS. 2 and 3, it can be appreciated that elastic members 120 provide a mechanism whereby sleeve 115 can be adjusted to closely conform to the diameter and shape of syringe barrel 210 by further overlapping the ends 125 of the adjustable sleeve 115 to reduce its inner diameter by allowing the stretched elastic members 120 to recoil and tighten around overlying sleeve 115.

In the embodiment shown in FIGS. 2 and 3, elastomeric members 110 are attached on either side of sleeve 115, preferably at distal end 130. In a preferred embodiment, elastomeric members 110 may be affixed at opposed sides of an elastic member 120

Once a loaded syringe is placed within sleeve 115, oriented so that plunger 205 is adjacent to cap 105, sleeve 115 is tightened about syringe barrel 210 and held in place by elastic members 120. Sleeve 115 is cylindrical being open at each end. Syringe 200 is held within sleeve 115 by the tension of sleeve 115 and is further prevented from sliding through sleeve 115 by barrel wings 203 of barrel 210 abutting against an upper end of sleeve 115. As seen in FIGS. 2 and 3, cap 105 is pulled away from sleeve 115 to a height greater than the height of plunger 205 so that cap 105 may clear and be placed upon plunger end 215. FIG. 3 schematically illustrates elastomeric members 110 in a highly stretched form.

FIG. 4 shows a preferred embodiment of the pump 100 in an operative position, after tightening of the sleeve 115 and positioning of the self-centering cap 105 over the end of the plunger 215. In a typical operative position, the elastomeric members 110 are stretched taut so as to generate force along the long axis of the plunger 205, in the direction of arrow B indicated in FIG. 4. As a result of the force and direction of arrow B, cap 105 centers itself relative to plunger 205. The force and direction of arrow B being consistent along the stroke length, which is at least as great as the length of plunger 205, is consistently applied along the stroke length.

As discussed above, existing IV administration systems and regulation devices are typically equipped with Luer lock attachments. The highly adjustable and open-ended nature of the adjustable body such as sleeve 115 of the pump 100 is compatible with all such connections, ensuring that use of multiple flow rates, pump durations and catheter choices is not limited by the design of the portable pump 100.

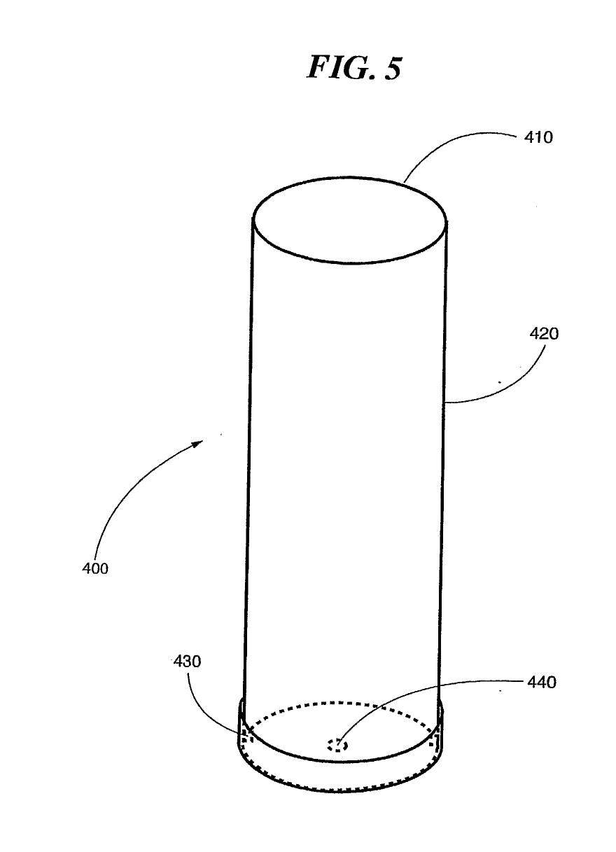

Some embodiments of the pumps are further provided with a safety cover, which is particularly useful in military, disaster-related or veterinary applications, for example. The safety cover prevents the patient or other physical interferences from disrupting delivery of the medicinal solution contained within the syringe to the patient. Referring to FIG. 5, in a preferred embodiment, the safety cover 400 comprises a first end 410 that is closed and a second end 420 that provides access to the pump, an IV exit port 440 and means 430 (e.g. a snap cap) for allowing entry and holding the syringe pump in place. A preferred safety cover is formed of transparent or translucent material, is unbreakable and rigid and fits syringe pumps of all sizes. The safety cover can be adapted to be attached by a strap or Velcro™ fastener, for example to a belt fanny pack or patient limb.

In another aspect, the invention provides various types of kits comprising one or more pump devices in accordance with the invention packaged along with one or more syringes pre-filled with a liquid medication, and instructions for use. The “bundled” kits can conveniently provide a wide range of liquid medications such as sterile pharmaceutical products including IV solutions such as saline, drug solutions, pain medications, antibiotics, hormone solutions such as insulin or human growth hormone, or blood products such as plasma or cells, along with the appropriate delivery means in the form of the pump device. Kits (both devices and medications) can be configured for both human and veterinary use, as required.

Configured as a self-contained self-powered pump device including an adjustable body, a single pump can accommodate the barrels of multiple sizes of syringes. By utilizing a concave member as the cap, a self-centering cap, adapted to fit many sizes and shapes of syringe plungers, is provided. Constructed of lightweight, inexpensive materials, the device can be manufactured at sufficiently low cost to permit disposable use, but is robust enough to withstand sterilization and multiple use if desired.

The inventive device is simple for patients and medical staff to understand and use. No drop counting or complex computer programming is required. In embodiments designed for use by patients at home or outside of hospital settings, clear instructions for use may be provided, for example in the form of universally understandable pictograms, either on the device itself (for example on the adjustable body) or in a package insert of a kit.

Lightweight, compact and discreet, the pump can be strapped to a patient with a suitable fastener such as a strip of Velcro, or it can be worn on a belt or under the clothing if desired. As discussed above, no cords or IV poles are required, allowing users to move about without restriction.

In contrast to other ambulatory designs, pumps in accordance with the invention are waterproof and completely submersible. Thus, patients can shower or bathe without taking special precautions.

Self-powered syringe pump devices in accordance with the invention are designed for use in all settings in which there is a need for infusion of a fluid medication over a period of time to a subject, such as a human patient. Some embodiments of pump devices within the scope of the invention comprise radiolucent materials, contain no metal parts, and produce no electronic emissions, making them suitable for use within large medical devices such as CT and MRI machines and hyperbaric chambers, as well as in air rescue applications. Accordingly, the inventive device has a wide variety of applications including but not limited to use in hospitals and medical facilities, medical vehicles such as ambulances and aircraft, medical care at home and in alternate sites, and remote use by military personnel and emergency responders.

The type of intravenous therapy that can be delivered by the device is not limited. As discussed in more detail below, typical applications include those in the areas of pain management, and infusion of chemotherapy and other intravenous drugs.

Advantageously for hospitals and medical service providers, portable pump devices in accordance with the invention provide both convenience and cost savings relative to existing infusion systems, and can be used to administer a wide spectrum of medications in both acute and chronic settings. Using the inventive device, physicians and other medical service providers may continuously infuse local anesthetic directly to a surgical site, and may provide accurate continuous or intermittent infusions of medications including but not limited to IV solutions, drug solutions, insulin, hormones, antibiotics, whole blood and packed red blood cells.

Some embodiments of the pump units are provided in sterile packages, and typically are sufficiently inexpensive to manufacture that they can be disposed after a single use. Nevertheless, the pumps are robustly constructed and alternatively can be easily cleaned and sterilized for multiple use, for example by sterilizing with alcohol, or autoclaving. Another important feature for safety and efficiency is compatibility with positive Luer locks, which are commonly used on IV administration systems to reduce the risk of inadvertent disconnection of the IV line by the patient or others.

Those patients capable of moving about in a hospital are able to benefit significantly from the freedom of movement provided by the ambulatory design of the device, which is completely free of cords, batteries or IV poles without concern about tripping over power cords or pole components. Patient self-sufficiency in moving about greatly benefits the hospital facility by reducing the need for nursing assistance for non-medical needs of patients.

Syringe pumps in accordance with the invention are ideal for use in MRI, CT, and hyperbaric chambers, comprising no metal parts or materials capable of producing electronic emissions. Accordingly, the pump does not interfere with devices that rely on radio frequencies or magnetic radiation for operation. The lack of metal or electronic parts in the pump (and syringe) allows infusion treatments to continue in hyperbaric chambers without risk of combustion.

For use in medical emergency rooms, preparation time for insertion of a patient IV is reduced due to fast, easy set-up of the pump and pre-filled syringes. The self-powering design of the pump eliminates the need to check or change a battery. Cost is further decreased because no heavy metal IV pole is required, and convenience to staff and patients is further improved by the absence of power cords or a cumbersome pole.

The invention provides many advantages for home and alternative care use around the world, in any application in which an IV unit is needed. The pump and syringe can be conveniently strapped to the patient to enable freedom of movement, a feature, which is particularly advantageous for children, as well as adult patients who require long periods of infusion of medication and do not wish to be tethered to their therapeutic delivery system. The universal fit of the adjustable body, and self-centering feature of the cap on preferred embodiments of the syringe pump provide for proper fit on various sizes of syringes in common use in different locations around the globe. Advantageously, the small cost of manufacture of the pump unit makes it feasible for suppliers of medication to bundle the pumps in a package along with syringes pre-loaded with medication, a feature that is also convenient for consumers.

One new and growing application of infusion therapy is in the area of ambulatory Patient Controlled Regional Analgesia (PCRA), by which patients deliver their own pain medication as needed. As the elder segment of the population continues to expand in the U.S. and in other developed nations, increasing numbers of patients require pain relief from chronic conditions such as arthritis, cancer, and diabetic and post-herpetic neuropathies. Infusion of pain medications by PCRA benefits patients by avoiding the side effects of oral medications and enabling pain relieving drugs to be delivered locally. The practice also shifts the burden of patient care sooner from institutions to individuals, reducing the duration of hospital stays. Other medications which increasingly are being infused in home care, alternative care and nursing care environments include chemotherapeutic agents, antibiotics, human growth hormone, insulin, and therapeutic agents for hemophilia and AIDS. As discussed, the inventive syringe pumps, being compact and self-contained, provide many advantages over existing pumps for use in chronic infusion settings outside of hospitals.

Aside from use by patients with chronic disorders, the inventive devices have many applications in myriad situations requiring emergency medical services delivered outside of medical facilities. Due to ease of use of the inventive pump, medication can be commenced promptly and easily while a patient is still in the field. As discussed, the pump device is lightweight, easily portable by personnel, ready to use and capable of delivering self-sufficient power on demand. Compatibility with pre-loaded syringes of any size, and fast, easy set-up enable the medication to be administered quickly in an emergency situation.

A particular advantage in ambulances and helicopter rescue situations is the fact that the patient and the pump travel as one unit, eliminating the need for additional personnel to manage the IV. Furthermore, elimination of a hanging drip bag overhead enables patients to occupy, and receive medical care, in smaller spaces than are needed with traditional IV poles and gravity-fed systems. Furthermore, the self-sufficient power source does not interfere with VHF radio frequencies used by aircraft, and the flow rate is not affected by changes in barometric pressure in aircraft.

The design features of syringe pumps in accordance with the invention are especially well adapted to military needs and uses. For medical applications in the field and in military hospitals, the inventive pumps provide the advantages of portability, light weight, and delivery of self-sufficient power on demand with no reliance on batteries. The pump device can be strapped to a patient, thereby eliminating the need for extra personnel to hold a drip bag.

Similarly, many features of the syringe pumps are ideal for situations involving emergencies and disaster relief, including use on victims of flooding and building collapse. Constructed of waterproof materials, the pumps continue to provide power even if wet or submerged. Generation of power to deliver the contents of the syringe can occur in any orientation of the pump, an aspect that is particularly advantageous for medicating subjects who cannot be moved and, for example, may be trapped in rubble or in a vehicle. Additionally, unlike battery-powered pumps, the inventive pump device can be stored indefinitely without losing power, making it particularly suitable for stockpiling in anticipation of future disasters.

Features of the pump device also make it suitable for use in applications in developing countries around the world in which existing medical facilities may have limited or no access to sources of electricity. As discussed, the low cost per unit for materials, lack of parts requiring ongoing maintenance or service and re-usability are particularly advantageous features of the invention. Similarly, diabetic patients in such locations who are unable to afford and/or run electrical or battery-powered insulin pumps can continuously infuse insulin using the inventive device.

Another use for self-powered syringe pump devices in accordance with the invention is to provide protection against acts of bioterrorism. For example, antidotes to chemical and biological warfare agents can be pre-loaded and stored in syringes. In the event of such an attack, the contents of the syringes can be delivered to a victim on demand at any location quickly and conveniently by emergency personnel. Large numbers of victims can be treated rapidly due to fast and easy set-up in any location, regardless of the availability of electrical power or batteries. Low cost to manufacture is a further benefit in this and many other situations described above, as is the option of disposability in situations in which likelihood of contamination is heightened.

In addition to human use, portable syringe pumps and kits in accordance with the invention are suitable for a wide variety of veterinary medical applications. Embodiments of the devices designed for veterinary use are adapted to be strapped to an animal subject, such as, for example, a horse, cow, pig, dog, cat, or bird, allowing for continuous delivery of medication without the need for constant supervision or restraint. The compact, lightweight design minimizes interference with the animal's movement and yet can withstand destruction, for example by crushing, due to construction from a resilient material (e.g., polycarbonate), and use of a safety cover if desired. The self-sufficient power source eliminates the need for batteries, or tethering to an electrical source. The waterproof nature of the materials enables an animal being treated, such as a horse or cow, to be housed outside if appropriate.

Those of skill in the art will recognize that there are many other applications for a self-powered portable pump device that can provide constant fluid flows through a syringe over an extended period of time. As but one non-limiting example, the device can be adapted for use in a cooling system to deliver a suitable bactericide, for example as a method of reducing the risk of spreading Legionnaire's disease.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A self-powered portable pump device for dispensing fluid content from a syringe, comprising:
a cap adapted to receive a plunger of a syringe;
an adjustable body adapted to hold the barrel of the syringe; and
at least two elastomeric members for operatively coupling the cap to the body, to apply a force to the plunger of the syringe for dispensing fluid content from the syringe.

2. The device of claim 1, wherein the cap, body and elastomeric members are fabricated from non-metallic materials.

3. The device of claim 2, wherein the non-metallic materials are plastic materials that can withstand sterilization with alcohol or by autoclaving.

4. The device of claim 1, wherein the adjustable body and sleeve are substantially transparent to electrical and magnetic signals.

5. The device of claim 1, wherein the cap is self-centering.

6. The device of claim 1, wherein the elastomeric members can return nearly 100% of the energy invested in them over the entire stroke length of the plunger.

7. The device of claim 1, wherein the adjustable body is formed from a sheet of material, said sheet of material being rolled upon itself to form a sleeve.

8. The device of claim 7, further comprising an elastic member disposed about said sleeve for maintaining an inner diameter of said sleeve and a diameter for holding the barrel of the syringe.

9. A self-powered portable pump device for dispensing fluid content from a syringe, comprising:
a self-centering cap adapted to receive a plunger of a syringe;
a body adapted to hold the barrel of the syringe; and
at least two elastomeric members for operatively coupling the cap to the body, to apply a force to the plunger of the syringe for dispensing fluid content from the syringe.

10. The device of claim 9, wherein the cap, body and elastomeric members are fabricated from materials substantially transparent to electronic and magnetic signals.

11. The device of claim 10, wherein the materials are plastic materials that can withstand sterilization with alcohol or by autoclaving.

12. The device of claim 9, wherein the elastomeric members return nearly 100% of the energy invested in them along the entire stroke length of the plunger.

13. The device of claim 9, wherein the self-centering cap has a convex shape.

14. The device of claim 13, wherein the shape is substantially symmetrical.

15. A self-powered portable pump device for dispensing fluid content from a syringe, comprising:
a cap adapted to receive a plunger of a syringe;
a body for holding the barrel of a syringe; and
at least two elastomeric members for operatively coupling the cap to the body, to apply a force to the plunger of the syringe for dispensing fluid content from the syringe, wherein the elastomeric members are fabricated from a polyurethane compound fortified with silicone having a tensile strength of about 700 psi (kg/cm).

16. The device of claim 15, wherein the cap is self-centering.

17. The device of claim 16, wherein the elastomeric members can return nearly 100% of the energy invested in them over the entire stroke length of the plunger.

18. The device of claim 15, wherein the body is adjustable.

19. The device of claim 18, wherein the adjustable body comprises an adjustable sleeve of flexible material rolled upon itself, with overlapping ends held in place by two or more elastic means.

20. A kit comprising the device of claim 1, and one or more syringes pre-filled with a liquid medication.

21. The kit of claim 20, wherein the liquid medication is a pharmaceutical product selected from an IV solution, a drug solution, a pain medication, an antibiotic, a hormone, or a blood product.

22. A kit comprising the device of claim 9, and one or more syringes pre-filled with a liquid medication.

23. The kit of claim 22, wherein the liquid medication is a pharmaceutical product selected from an IV solution, a drug solution, a pain medication, an antibiotic, a hormone, or a blood product.

24. A kit comprising the device of claim 15, and one or more syringes pre-filled with a liquid medication.

25. The kit of claim 24, wherein the liquid medication is a pharmaceutical product selected from an IV solution, a drug solution, a pain medication, an antibiotic, a hormone, or a blood product.

26. A safety cover adapted to fit the device of any of claims 1, 9, and 15.
